(19) United States
(12) Patent Application Publication
(10) Pub. No.: US 2014/0343105 A1
(43) Pub. Date: Nov. 20, 2014

(54) NOVEL ORALLY ADMINISTERED DABIGATRAN FORMULATION

(71) Applicant: Georg BOECK, Laupheim (DE)

(72) Inventor: Georg BOECK, Laupheim (DE)

(73) Assignee: BOEHRINGER INGELHEIM INTERNATIONAL GMBH, Ingelheim am Rhein (DE)

(21) Appl. No.: 14/452,703

(22) Filed: Aug. 6, 2014

Related U.S. Application Data
(63) Continuation of application No. 13/747,563, filed on Jan. 23, 2013, now abandoned.

(30) Foreign Application Priority Data
Jan. 24, 2012 (EP) 12152327.8

(51) Int. Cl. A61K 47/48; A61K 31/4439

(52) U.S. Cl. CPC A61K 47/48969; A61K 31/4439
USPC 514/338

(57) ABSTRACT

The invention relates to a new medicament formulation of the active substance dabigatran etexilate of formula I

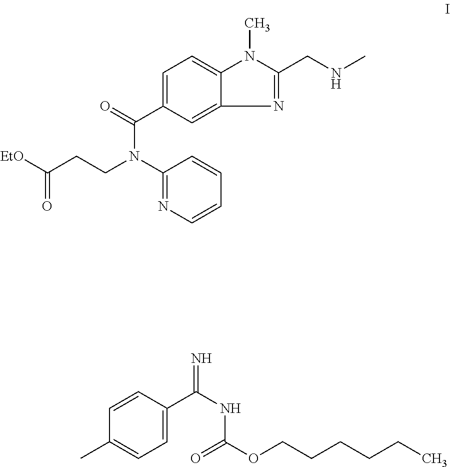

optionally in the form of the pharmaceutically acceptable salts thereof, as well as the preparation thereof.

The invention relates to a new medicament formulation of the active substance dabigatran etexilate of formula I

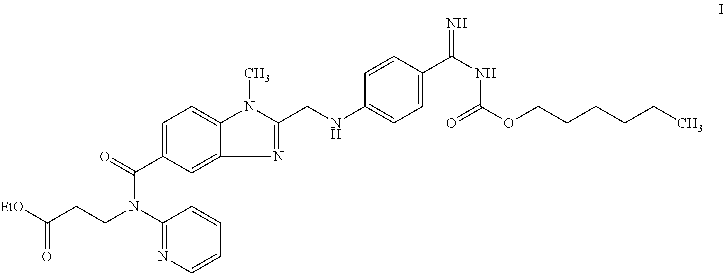 [SMILES: CCCCCCOC(=O)NC(=N)c1ccc(NCc2nc3cc(C(=O)N(CCC(=O)OCC)c4ccccn4)ccc3n2C)cc1]

optionally in the form of the pharmaceutically acceptable salts thereof, as well as the preparation thereof.

### BACKGROUND TO THE INVENTION

The compound of formula I is known from the prior art and was first disclosed in WO98/37075. It is a potent thrombin inhibitor which can be used for example for the post-operative prevention of deep vein thromboses and in stroke prevention, particularly for preventing strokes in patients with atrial fibrillation. WO 03/074056 discloses the methanesulphonic acid addition salt of dabiagtran etexilate (i.e: dabigatran etexilate methansulphonate) to be particularly useful. According to WO 05/028468 the methansulphonic acid addition salt of dabiagtran etexilate exists in different polymorphic forms.

The compound is usually administered orally. In particular, so-called pellet formulations are used, as disclosed for example in WO 03/074056. Due to the poor water solubility of Dabigatran etexilate under ambient temperature it is necessary to administer the compound in administration forms improving the solubility and bioavailability thereof.

Cyclodextrins are known as excipients in preparing pharmaceutical formulations of certain active ingredients for improving solubility (“Cyclodextrins as pharmaceutical solubilizers”; Brewster, Loftsson; Advanced Drug Delivery, 2007 (59): 645-666) or bioavailability (“Cyclodextrins in drug delivery”; Loftsson, Jarho et al.; Expert Opinion Drug Delivery; 2005, 2(2): 335-351) of the active ingredient.

The aim of the present invention is to provide an alternative solid or liquid medicament formulation, i.e a pharmaceutical composition, for oral administration of dabiagtran etexilate or a pharmaceutically acceptable acid addition salt thereof, particularly dabigatran etexilate methansulphonate.

### DESCRIPTION OF THE FIGURES

FIG. 1a Drug load DS/CD [mol/mol] measured for various types of cyclodextrins at various pH values for the CD concentration of 10% (w/v).

FIG. 1b Drug load DS/CD [g/g] measured for various types of cyclodextrins at various pH values for the CD concentration of 10% (w/v).

FIG. 2a Intrinsic Solubility at different pH values without cyclodextrins

FIG. 2b Solubility of DS [mg/ml] measured for various types of cyclodextrins at various pH values for the CD concentration of 10% (w/v).

FIG. 3a Influence of concentration of CMC-Na on the complexation with α-CD (10% (w/v))

FIG. 3b Solubility of DS relative to the concentration of α-CD with CMC-Na (0.25% (w/v)) at pH 3.

### DETAILED DESCRIPTION OF THE INVENTION

The present invention relates to an oral pharmaceutical composition comprising a compound of formula I, i.e. dabigatran etexilate, or a pharmaceutically acceptable salt, solvates, hydrates or combinations thereof,

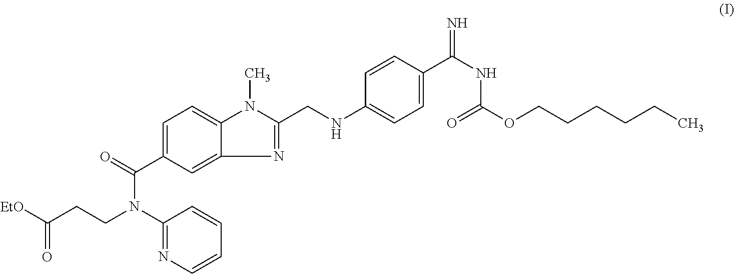 [SMILES: CCCCCCOC(=O)NC(=N)c1ccc(NCc2nc3cc(C(=O)N(CCC(=O)OCC)c4ccccn4)ccc3n2C)cc1]

and as excipient at least one water soluble cyclodextrin agent.

Preferred is a pharmaceutical composition wherein the active ingredient is dabigatran etexilate methanesulphonate. Also preferred is a pharmaceutical composition wherein the cyclodextrin is a water soluble cyclodextrin. Also preferred is a pharmaceutical composition wherein the cyclodextrin is selected from the group consisting of α-Cyclodextrin (α-CD), β-Cyclodextrin (β-CD), γ-Cyclodextrin (γ-CD), Hydroxypropyl-β-Cyclodextrin (HP-β-CD), Methyl-β-Cyclodextrin (M-β-CD), Hydroxypropyl-γ-Cyclodextrin (HP-γ-CD) and Sulfobuthylether-β-Cyclodextrin (SBE-β-CD), preferably selected from the group consisting of α-CD, M-β-CD and HP-β-CD, more preferably α-CD and M-β-CD or a mixture thereof. Particularly preferred is a pharmaceutical composition wherein the cyclodextrin is α-Cyclodextrin.

Also preferred is a pharmaceutical composition wherein the cyclodextrin is from 20 to 99 wt %, preferably 30 to 90 wt %, more preferably 50 to 80 wt % of the composition. Also preferred is a pharmaceutical composition wherein the compound of formula I is from 1 to 80 wt %, preferably 10 to 70 wt %, more preferably 20 to 50 wt %, of the composition.

A composition according to the current invention may further comprise one or more excipients of the group consisting of preservatives, antioxidants, stabilizers, colorants, sweeteners and flavorants, or mixtures thereof.

Particularly preferred is a pharmaceutical composition wherein the antioxidant is selected from the group consisting of BHA, BHT, EDTA and Propylgallate.

Particularly preferred is a pharmaceutical composition wherein the sweetener is selected from the group consisting of sugars, e.g. glucose, glucose sirup, fructose, saccharose, maltose or lactose, alcohols or, sugar substitutes.

Also preferred is a pharmaceutical composition further comprising a hydrophilic polymer. Particularly preferred is a pharmaceutical composition wherein the hydrophilic polymer is selected from the group consisting of methyl cellulose, hydroxethyl cellulose, hydroxypropyl cellulose, hydroxypropylmethyl cellulose, carboxymethyl cellulose, povidone and polyethyleneglycol, preferably selected from the group consisting of povidone and Hydroxyethyl cellusols, more preferably hydroxypropyl cellulose and methyl cellulose, most preferably carboxymethyl cellulose (CMC)-Na. Commercially available hydrophilic polymers which may be used by way of example are Methocel E15LV, Methocel A4C, PEG 6000, Kollidon K25, Klucel, Natrosol HX 250 and CMC-Na (Blanose™).

Also preferred is a pharmaceutical composition wherein the composition is consisting of a compound according to formula I or a pharmaceutically acceptable salt, solvates, hydrates or combinations thereof, at least one cyclodextrin agent, optionally a solvent and optionally a hydrophilic polymer, preferably a compound according to formula I or a pharmaceutically acceptable salt, solvates, hydrates or combinations thereof, at least one cyclodextrin agent, a solvent and a hydrophilic polymer, more preferably a compound according to formula I or a pharmaceutically acceptable salt, solvates, hydrates or combinations thereof, at least one cyclodextrin agent and a solvent. Also preferred is a pharmaceutical composition wherein the composition is consisting of a compound according to formula I or a pharmaceutically acceptable salt, solvates, hydrates or combinations thereof and at least one cyclodextrin agent.

In another aspect the current invention relates to an inclusion complex in aqueous solution comprising a compound of formula I or a pharmaceutically acceptable salt thereof and a water soluble cyclodextrin.

Preferred is an inclusion complex wherein said salt is the methanesulphonate salt. Also preferred is an inclusion complex wherein the drug load (DS/CD) is from 0.1 to 0.9 [g/g] more preferably from 0.15 to 0.8 [g/g], most preferably from 0.2 to 0.7 [g/g].

The invention further relates to a process which allows the formulation to be manufactured on a large scale with a reproducible quality. Preferred is a process for preparing a composition which comprises

a) preparing an aqueous optionally buffered solvent in the pH range of pH 1 to pH 14, more preferably in the range of 2 to 4, particularly preferred of 2.5 to 3.5, most preferably pH 3.

b) adding a water soluble cyclodextrin to the solvent, preferably 1 to 99 g cyclodextrin per 100 ml solvent, more preferably 10 to 50 g/100 ml, and most preferably 20 to 40 g/100 ml.

c) stirring the mixture until the mixture becomes clear,

d) optionally adding a hydrophilic polymer to the mixture, preferably 0.01 g to 20 g hydrophilic polymer per 100 ml solvent, more preferably 0.01 g to 5 g hydrophilic polymer per 100 ml solvent, particularly preferred 0.05 to 0.5 g hydrophilic polymer per 100 ml solvent,

e) adding a compound of formula I or a pharmaceutically acceptable salt thereof, preferably 0.01 mg to 300 mg DS per 1 ml solvent, more preferably 0.1 mg to 200 mg DS per 1 ml solvent, particularly preferred 1 to 130 mg DS per 1 ml solvent and

f) stirring the mixture until the mixture becomes clear

while steps a) to f) take place successively in the order stated.

Also preferred is a process wherein the pH of the aqueous solution is from 2 to 4, preferably from 2.5 to 3.5, most preferably pH 3.

All process steps a) to f) are carried out at an elevated temperature, preferably at a temperature in the range from 4° C. to 60° C., particularly preferred in the range from 20° C. to 30° C.

Also preferred is a process further comprising steps g) and h), wherein

g) denotes drying of the solution, e.g. spray drying, lyophilisation, spray-coating onto pellets and spray-granulation.

h) denotes further processing of the resulting powder into solid forms selected from the group consisting of tablets, capsules, pellets, powder for reconstitution and extended release solid formulations, preferably selected from the group consisting of powder for reconstitution, capsules, tablets and pellets, more preferably tablets and capsules, most preferably tablets.

The compound of formula I is referred to as “active ingredient”, i.e. the free active substance base (DS), hereinbefore and hereinafter.

Preferably according to the invention the acid addition salts of the active ingredient are selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate, preferably hydromethanesulphonate.

According to WO 05/028468 the methansulphonic acid addition salt of dabiagtran etexilate exhibits polymorphism. Preferably anhydrous Polymorph I, anhydrous Polymorph II or the hemihydrate of the methansulphonic acid addition salt of dabiagtran etexilate may be used. Polymorph I is the most preferred polymorph.

The term water soluble cyclodextrin agent as used herein is defined as a pharmaceutically acceptable excipient having a water solubility of at least 1 g/100 ml at 25°, preferably 10 to 60 mg/ml at 25°, which is a cyclic oligosaccharide comprising 6, 7 or 8 alpha-D-glucopyrannose units, i.e. alpha, beta or gamma cyclodextrin respectively. Preferably, the cyclodextrin agent is selected from the group consisting of α-Cyclodextrin (α-CD), β-Cyclodextrin (β-CD), γ-Cyclodextrin (γ-CD), Hydroxypropyl-β-Cyclodextrin (HP-β-CD), Methyl-β-Cyclodextrin (M-β-CD), Hydroxypropyl-γ-Cyclodextrin (HP-γ-CD) and Sulfobuthylether-β-Cyclodextrin (SBE-β-CD), preferably selected from the group consisting of α-CD, M-β-CD and HP-β-CD, more preferably α-CD and M-β-CD or a mixture thereof, most preferably α-CD.

Examples of solvents which may be used in the process for preparing a composition according to the invention may be selected from among water, hydrochloric acid, preferably 0.001 to 0.1 N hydrochloric acid, Soerensen buffer solution, particularly preferred is 0.001 N hydrochloric acid. The invention further relates to a pharmaceutical composition according to the invention administered to a mammal in need thereof with a daily dose of 10 mg to 300 mg DS, preferably 75 mg to 150 mg DS, preferably in the form of Dabigatran etexilate methanesulphonate. The invention further relates to a pharmaceutical composition for the post operative prevention of deep vein thromboses and stroke prevention, particularly for preventing strokes in patients with atrial fibrillation.

The pharmaceutical composition may be administered in form of a solution or in solid form. Preferably said solution contains 0.01 mg/ml to 300 mg/ml DS, preferably 1 mg/ml to 130 mg/ml DS, preferably in the form of Dabigatran etexilate methanesulphonate. The compositions according to the invention may be prepared analogously to the following examples. The Examples described below are intended as an illustration of the invention without restricting it.

### EXAMPLES

1. Solutions

1.1 Solution 1

Composition

| Ingredients | Amount | |
|---|---|---|
| α-CD | 14 | g |
| Dabigatranetexilat | 0.673 | g |
| methanesulphonate⁽¹⁾ | | |
| CMC-Na | 0.25 | g |
| Hydrochlorid acid 0.001N | Ad 100 | ml |

⁽¹⁾Corresponds to 0.584 g of free active substance base (DS) (conversion factor 1.153)

Preparation:

About 60 to 70 ml of the 0.001 N HCl is filled into a beaker and heated to about 60° C. The water content of the cyclodextrin needs to be taken into account for the preparation. 14 g α-Cyclodextrin (calculated to the dry mass) is dispersed slowly to the solvent and stirred until a clear solution is reached. Then, 0.25 g CMC-Na is added slowly while stirring. The solution is let cool down to room temperature and filled up to about 90 ml. The solution is stored for about 12 hours at room temperature. The pH value of the solution is analysed and adjusted, if necessary. The solution is quantitatively transferred into a volumetric flask. The volumetric flask is filled up to the labeling.

1.2 Solution 2

Composition

| Ingredients | Amount | |
|---|---|---|
| HP-β-CD | 50 | g |
| Dabigatranetexilat | 0.985 | g |
| methanesulphonate⁽¹⁾ | | |
| CMC-Na | 0.25 | g |
| Hydrochlorid acid 0.001N | Ad 100 | ml |

⁽¹⁾Corresponds to 0.854 g of free active substance base (DS) (conversion factor 1.153)

Preparation

About 60 to 70 ml of the 0.001 N HCl is filled into a beaker and heated to about 60° C. The water content of the cyclodextrin needs to be taken into account for the preparation. 40 g HP-β-Cyclodextrin (calculated to the dry mass) is dispersed slowly to the solvent and stirred until a clear solution is reached. Then, 0.25 g CMC-Na is added slowly while stirring. The solution is let cool down to room temperature and filled up to about 90 ml. The solution is stored for about 12 hours at room temperature. The pH value of the solution is analysed and adjusted, if necessary. The solution is quantitatively transferred into a volumetric. The volumetric flask is filled up to the labeling.

1.3 Solution 3

Composition

| Ingredients | Amount | |
|---|---|---|
| M-β-CD | 40 | g |
| Dabigatranetexilat | 1.232 | g |
| methanesulphonate⁽¹⁾ | | |
| CMC-Na | 0.25 | g |
| Hydrochlorid acid 0.001N | Ad 100 | ml |

⁽¹⁾Corresponds to 1.069 g of free active substance base (DS) (conversion factor 1.153)

Preparation:

About 60 to 70 ml of the 0.001 N HCl is filled into a beaker and heated to about 60° C. The water content of the cyclodextrin needs to be taken into account for the preparation. 40 g Methyl-β-Cyclodextrin (calculated to the dry mass) is dispersed slowly to the solvent and stirred until a clear solution is reached. Then, 0.25 g CMC-Na is added slowly while stirring. The solution is let cool down to room temperature and filled up to about 90 ml. The solution is stored for about 12 hours at room temperature. The pH value of the solution is analysed and adjusted, if necessary. The solution is quantitatively transferred into a volumetric. The volumetric flask is filled up to the labeling.

1.4 Solution 4

Composition

| | |
|---|---|
| Ingredients | Amount |

| | | |
|---|---|---|
| SBE-β-CD | 50 | g |
| Dabigatranetexilat | 0.947 | g |
| methanesulphonate⁽¹⁾ | | |
| CMC-Na | 0.25 | g |
| Hydrochlorid acid 0.001N | Ad 100 | ml |

⁽¹⁾Corresponds to 0.821 g of free active substance base (DS) (conversion factor 1.153)

Preparation:

About 60 to 70 ml of the 0.001 N HCl is filled into a beaker and heated to about 60° C. The water content of the cyclodextrin needs to be taken into account for the preparation. 40 g Sulfobuthylether-β-Cyclodextrin (calculated to the dry mass) is dispersed slowly to the solvent and stirred until a clear solution is reached. Then, 0.25 g CMC-Na is added slowly while stirring. The solution is let cool down to room temperature and filled up to about 90 ml. The solution is stored for about 12 hours at room temperature. The pH value of the solution is analysed and adjusted, if necessary. The solution is quantitatively transferred into a volumetric. The volumetric flask is filled up to the labeling.

2. Capsule

a) Cyclodextrin Solution:

Composition

| Ingredients | Amount | |
|---|---|---|
| α-CD | 56 | g |
| Dabigatranetexilat | 2.256 | g |
| methanesulphonate⁽¹⁾ | | |
| CMC-Na | 1 | g |
| Hydrochlorid acid 0.001N | Ad 400 | ml (pH 3) |

⁽¹⁾Corresponds to 1.957 g of free active substance base (DS) (conversion factor 1.153)

Preparation:

About 240 to 280 ml of the 0.001 N HCl is filled into a beaker and heated to about 60° C. The water content of the cyclodextrin needs to be taken into account for the preparation. 56 g α-Cyclodextrin (calculated to the dry mass) is dispersed slowly to the solvent and stirred until a clear solution is reached. Then, 1 g CMC-Na is added slowly while stirring. The solution is let cool down to room temperature and filled up to about 360 ml. The solution is stored for about 12 hours at room temperature. The pH value of the solution is analysed and adjusted to pH 3, if necessary. The solution is quantitatively transferred into a volumetric. The volumetric flask is filled up to the labeling.

b) Spray Drying

The cyclodextrin solution was spray dried with the Büchi Labortechnik GmBH Spray-Dryer B290 using the following parameters:

| Inlet temperature | 140° | C. |
|---|---|---|
| Aspirator | 75% | |
| Pump | 5% (corresponds to | |
| | about 2.5 ml/min) | |
| Cleaning of nozzle | 1 | unit |
| Pressure | 30 | units |
| Nozzle cap | 1.5 | mm |
| Nozzle | 0.7 | mm |

| | |
|---|---|
| Cyclone | Standard |

c) Granulation

Composition:

| Ingredients | Amount |
|---|---|
| Spray-dried complex | 13 g |
| Tablettose | 3.25 g |
| 3% (w/v) Povidone solution | 10 ml |

Prior to the granulaton the spray-dried product together with Tablettose is homogeneously blended by means of a Turbula blender (speed: 32 units) for about 30 minutes. Batches of about 11 to 15 g of the spray-dried product are granulated together with about 20% (w/w) of Tablettose with about 10 ml of 3% (w/v) aqueous or isopropyl alcohol containing polyvinylpyrrolidon solution. The granulation is performed with a Diosna-Pharma Mixer P1/6 equipped with a 250 ml bowl using the following parameters:

| Granulation program | Rotor speed [rpm] | Chopper speed [rpm] |
|---|---|---|
| 30 sec. pre-blending | 600 | 750 |
| 30 sec. addition of | 600 | 750 |
| granulation fluid | | |
| 30 sec. granulation | 1200 | 1500 |

After the granulation the mass was wet sieved through a 1600 μm sieve and dried in a vacuum oven at 50° C./25 mbar for about 20 hours. Finally, a dry sieving step with an 800 μm sieve was performed.

d) Capsule Filling

The granules were manually filled into hard gelatin capsules of different sizes, depending on the amount of DS needed.

Dabigatranetexilat methanesulphonate is tested for its solubility in dependency of the presence of various cyclodextrins at various ph values as follows:

### Methods to Investigate the Solubility of the Active Ingredient (DS)

2 ml Eppendorf tubes can be filled with about 1.8 ml aqueous solution with a selected pH value. The volume used for the screening is 1.8 ml. The molar amount of cyclodextrin (CD) in the given volume is calculated for the given CD concentration. For a given molar ratio of active ingredient DS/CD the amount of active ingredient (DS) is calculated. Normally, a molar ratio of DS/CD=1/1 is targeted: 1 mol DS and 1 mol CD. As precise as possible, the calculated amount of DS is weighted into the tube exactly. The actual amount is reported.

According to the actual amount of DS the necessary volume of CD solution is calculated. As precise as possible, the requested volume of CD solution is pipetted into the tube. The actual volume pipetted is reported. The solution in the tube is placed on an Eppendorf Vario shaker. The solution is intensively shaken by means of ultrasound (Hielscher Ultrasound Processor) for 10 seconds at the maximum. It is payed attention to heat generation and overheating has to be avoided.

The tube is shaken for 1 hour in the Eppendorf Thermomixer at the given temperature and at 800 rpm. Subsequently, the pH value is checked and the given pH value adjusted accordingly, if necessary. The tube is shaken for 24 hours in the Eppendorf Thermomixer at the given temperature and at 800 rpm.

Eppendorf tubes are centrifuged for about 15 minutes at the given temperature, but at 40° C. at the maximum (max. temperature of the centrifuge), and 14,000 rpm. In case no clear supernatant is obtained centrifugation is repeated with adjusted rpm (revolutions per minute) and time. In case the supernatant is not clear and a filtration step is necessary the content of the tube is sucked or poured into 2 ml single use syringe. The supernatant is filtered through a 0.2 μm filter (Pall GHP Acrodisc 13 mm Syringe Filter 0.2 μm pore size). About 2-3 drops of the solution are discarded. The rest of the volume is collected in a second tube. If temperatures above room temperature (about 22° C.) are given, filters and tubes have to be tempered at that temperature prior to use.

The clear supernatant or filtrate is used for the analysis of the concentration. The pH value of the clear solution is analysed. The DS concentration of the clear solution is measured by UV/VIS spectroscopy (e.g., Hewlett-Packard 8453 UV/Visible Spectrophotometer).

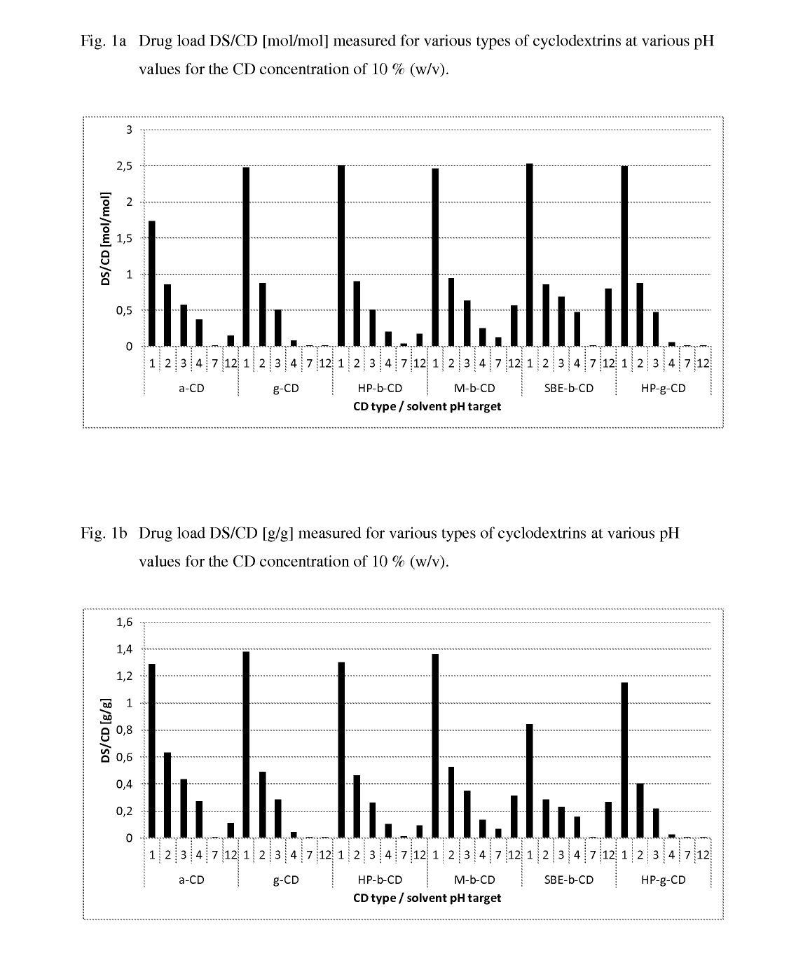

The drug load DS/CD is calculated for different cylodextrins at various pH values and shown in FIG. 1a and in FIG. 1b

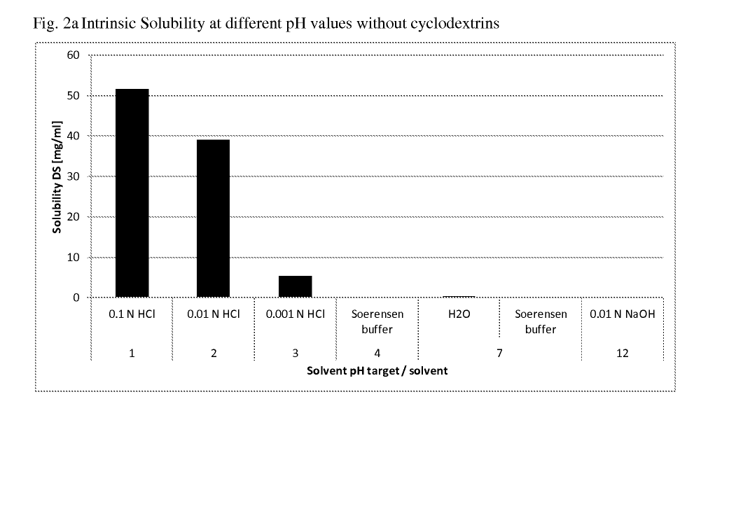

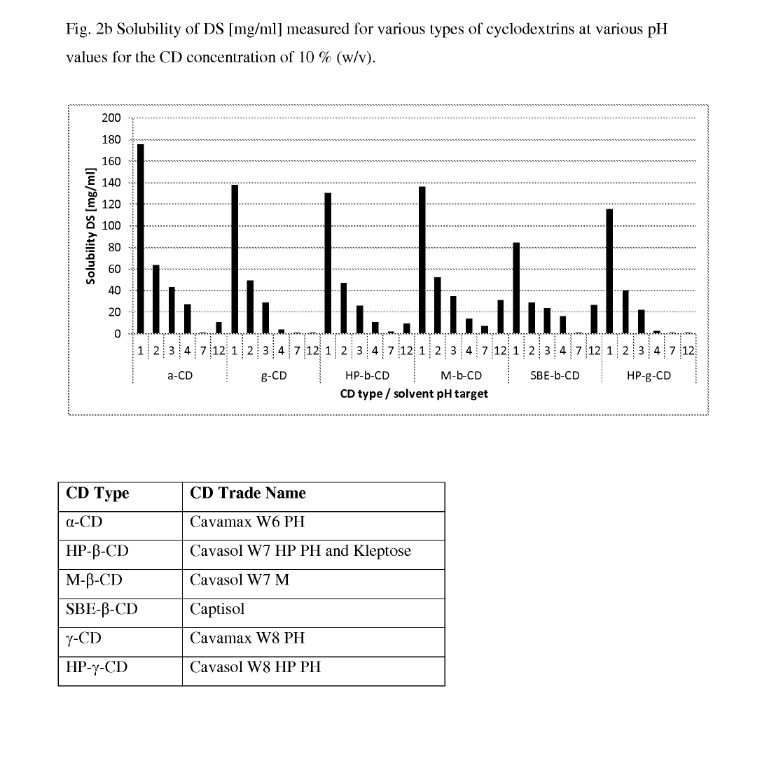

As a result from the method described above surprisingly all cyclodextrins are suitable to significantly improve the solubility of the active ingredient. In FIG. 2a the intrinsic solubility of the DS is shown. In FIG. 2b the solubility of the DS in dependency of solvent pH and cyclodextrin type is shown. For example with α-CD a solubility of about 43 mg/ml can be achieved by using a 10% (w/v) CD solution at pH 3. About 1.7 mol α-CD (for 10% (w/v)) are necessary to complex 1 mol API (DS/CD≈0.58 [mol/mol] or ≈0.43 [g/g].

DS, especially Dabigatranetexilat methanesulphonate, is tested for its solubility in dependency of the presence of various hydrophilic polymers and various cyclodextrins whereby the hydrophilic polymer is added to the aqueous solution before adding the DS. As a result from the method described above surprisingly it was found that the complexation efficiency of the CD is significantly improved by addition of a hydrophilic polymer.

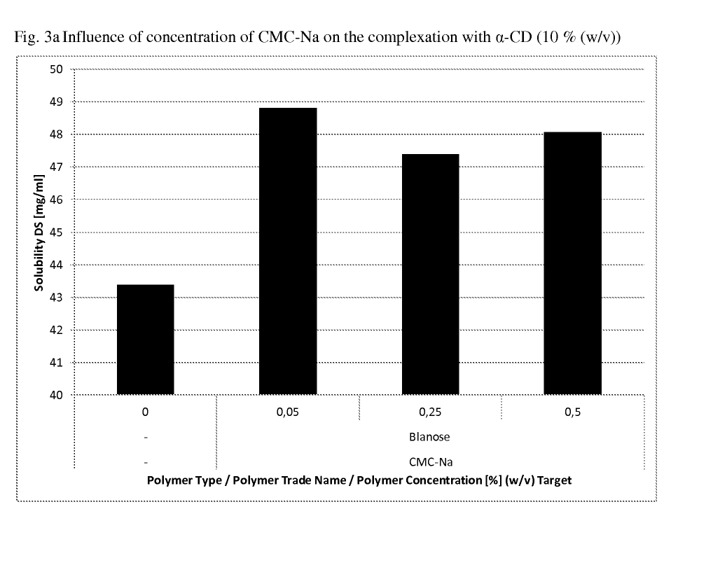

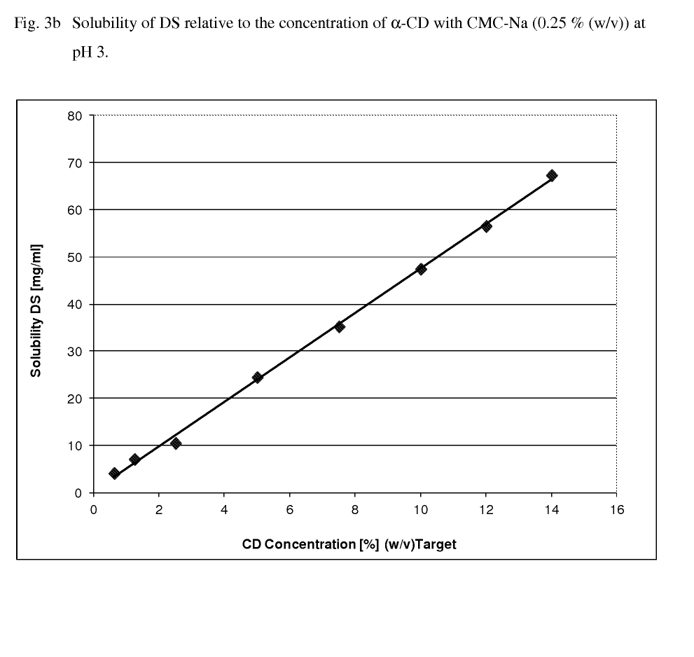

For example, as illustrated in FIG. 3a, the addition of 0.05% to 0.5% of CMC-Na leads to an improvement of the complexation efficiacy, i.e. an increase of the solubility of the DS and of the drug load DS/CD up to 11%. For example with α-CD a solubility of about 67.3 mg/ml can be achieved by using a 14% α-CD solution at pH 3 with 0.25% CMC-Na as illustrated in FIG. 3b. About 1.6 mol α-CD are necessary to complex 1 mol API (DS/CD≈0.63 [mol/mol]) or ≈2.1 g α-CD to complex 1 g API (DS/CD≈0.47 [g/g]) when CMC-Na is added.

1. Pharmaceutical composition for oral administration comprising a compound of formula I or a pharmaceutically acceptable salt or combinations thereof 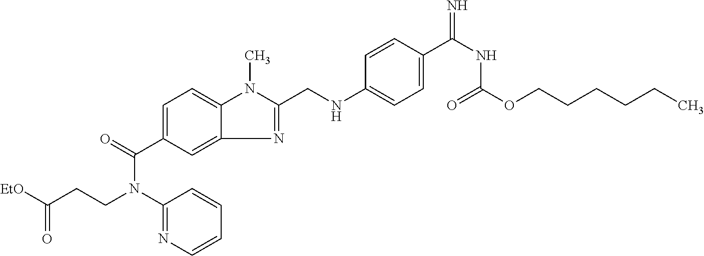 [SMILES: CCCCCCOC(=O)NC(=N)c1ccc(NCc2nc3cc(C(=O)N(CCC(=O)OCC)c4ccccn4)ccc3n2C)cc1]
and as excipient at least one cyclodextrin agent.

2. The composition according to claim 1 characterized in that the cyclodextrin is a water soluble cyclodextrin.

3. Pharmaceutical composition according to claim 1 characterized in that the active ingredient is dabigatran etexilate methanesulphonate.

4. The composition according to claim 1 characterized in that the cyclodextrin is selected from the group consisting of α-Cyclodextrin (α-CD), β-Cyclodextrin (β-CD), γ-Cyclodextrin (γ-CD), Hydroxypropyl-β-Cyclodextrin (HP-β-CD), Methyl-β-Cyclodextrin (M-β-CD), Hydroxypropyl-γ-Cyclodextrin (HP-γ-CD) and Sulfobuthylether-β-Cyclodextrin (SBE-β-CD), or a mixture thereof.

5. The composition according to claim 4 characterized in that the cyclodextrin is α-Cyclodextrin.

6. The composition according to claim 1 characterized in that the cyclodextrin is from 20 to 99 wt % of the composition.

7. The composition according to claim 1 characterized in that the compound of formula I is from 1 to 80 wt % of the composition.

8. The composition according to claim 1 further comprising a hydrophilic polymer.

9. The composition according to claim 8 characterized in that the hydrophilic polymer is selected from the group consisting of methyl cellulose, hydroxethyl cellulose, hydroxypropyl cellulose, hydroxypropylmethyl cellulose, carboxymethyl cellulose, povidone and polyethyleneglycol.

10. The composition according to claim 1 characterized in that the composition is consisting of a compound according to formula I or a pharmaceutically acceptable salt or combinations thereof, at least one cyclodextrin agent and optionally a hydrophilic polymer.

11. An inclusion complex in aqueous solution comprising a compound of formula I according to claim 1 or a pharmaceutically acceptable salt thereof and a water soluble cyclodextrin.

12. An inclusion complex according to claim 11 characterized in that said salt is the methanesulphonate salt.

13. An inclusion complex according to claim 11 characterized in that the drug load is 1% to 80% (w/w).

14. A process for preparing a composition according to claim 1 which comprises
a) preparing an aqueous optionally buffered solvent in the pH range of pH 1 to pH 14
b) adding a water soluble cyclodextrin to the solvent,
c) stirring the mixture until the mixture becomes clear,
d) optionally adding a hydrophilic polymer to the mixture,
e) adding a compound of formula I or a pharmaceutically acceptable salt thereof, and
f) stirring the mixture until the mixture becomes clear
while steps a) to f) take place successively in the order stated.

15. A process according to claim 14 further comprising steps g) and h), wherein
g) denotes drying of the solution and
h) denotes further processing of the resulting powder into solid forms selected from the group consisting of tablets, capsules, pellets, powder for reconstitution and extended release solid formulations.
